(19) United States
(12) Patent Application Publication
(10) Pub. No.: US 2020/0121501 A1
(43) Pub. Date: Apr. 23, 2020

(54) LASER TREATMENT APPARATUS AND METHOD FOR CONTROLLING THE SAME

(71) Applicant: TOPCON CORPORATION, Tokyo (JP)

(72) Inventors: Tatsuo YAMAGUCHI, Warabi-shi (JP); Kazuhiro OMORI, Setagaya-ku (JP)

(73) Assignee: TOPCON CORPORATION, Tokyo (JP)

(21) Appl. No.: 16/724,309

(22) Filed: Dec. 22, 2019

Related U.S. Application Data
(63) Continuation of application No. PCT/JP2018/021306, filed on Jun. 4, 2018.

(30) Foreign Application Priority Data
Jun. 29, 2017 (JP) 2017-126868

(51) Int. Cl. A61F 9/008

(52) U.S. Cl. CPC A61F 9/008; A61B 2018/20553; A61F 2009/00851

(57) ABSTRACT

A laser treatment apparatus includes an irradiation system, a wavefront changing unit, and a controller. The irradiation system is configured to output laser light for treatment from a light source. The wavefront changing unit is configured to change a wave front of the laser light for treatment output by the irradiation system to guide the laser light for treatment whose wave front is changed to a patient's eye. The controller is configured to control the wavefront changing unit.

### CROSS-REFERENCE TO RELATED APPLICATIONS

The present application is a continuation application of International Patent Application No. PCT/JP2018/021306, filed Jun. 4, 2018, which claims priority to Japanese Patent Application No. 2017-126868, filed Jun. 29, 2017. The contents of these applications are incorporated herein by reference in their entirety.

### FIELD

The disclosure relates to a laser treatment apparatus and a method for controlling the same.

### BACKGROUND

Laser treatment apparatuses for ophthalmology are used for photocoagulation of eye tissue, photoablation of eye tissue, and the like. In the conventional laser treatment apparatuses, the laser light is aimed while observing the front image of the eye using an observation apparatus such as a slit lamp microscope or a surgical microscope. In recent years, a laser treatment apparatus incorporating an optical coherence tomography apparatus (OCT apparatus) capable of acquiring a tomographic image of the eye has also appeared.

For example, Japanese Unexamined Patent Application Publication No. 2015-058152 discloses the laser treatment apparatus incorporating the OCT apparatus. This laser treatment apparatus is configured to reliably perform tomographic measurement at an irradiated target site of a patient's eye, by arranging an optical path of an irradiation optical system for irradiating the patient's eye with laser light and an optical path of an interference optical system substantially coaxially.

### SUMMARY

One aspect of some embodiments is a laser treatment apparatus, including: an irradiation system configured to output laser light for treatment from a light source; a wavefront changing unit configured to change a wave front of the laser light for treatment output by the irradiation system to guide the laser light for treatment whose wave front is changed to a patient's eye; and a controller configured to control the wavefront changing unit.

Another aspect of some embodiments is a method of controlling a laser treatment apparatus, including: an irradiation step of outputting laser light for treatment from a light source; a wavefront changing step of changing a wave front of the laser light for treatment output in the irradiation step to guide the laser light for treatment whose wave front is changed to a patient's eye, based on light receiving result of returning light from the patient's eye obtained by irradiating the patient's eye with light.

### BRIEF DESCRIPTION OF THE DRAWINGS

FIG. 1 is a schematic diagram illustrating a configuration example of a laser treatment apparatus according to embodiments.

FIG. 2 is a schematic diagram illustrating an operation example of the laser treatment apparatus according to the embodiments.

FIG. 3 is a schematic diagram for explaining an operation of the laser treatment apparatus according to the embodiments.

FIG. 4 is a schematic diagram illustrating a configuration example of the laser treatment apparatus according to the embodiments.

FIG. 5 is a schematic diagram illustrating a configuration example of the laser treatment apparatus according to the embodiments.

FIG. 6 is a schematic diagram for explaining an operation of the laser treatment apparatus according to the embodiments.

FIG. 7 is a schematic diagram for explaining an operation of the laser treatment apparatus according to the embodiments.

FIG. 8 is a schematic diagram for explaining an operation of the laser treatment apparatus according to the embodiments.

FIG. 9 is a schematic diagram illustrating a configuration example of the laser treatment apparatus according to the embodiments.

FIG. 10 is a schematic diagram illustrating a configuration example of the laser treatment apparatus according to the embodiments.

### DETAILED DESCRIPTION

In the laser treatment apparatuses, when the laser light cannot be sufficiently collected, the laser light is also irradiated around the irradiated target site. For example, when the irradiated target site is the retinal pigment epithelium layer, the laser light is also irradiated to a site other than the retinal pigment epithelium layer. Thereby, a tissue of the site other than the retinal pigment epithelium layer may be damaged. In particular, when the irradiated target site is in the vicinity of the fovea of the retina, the cone may be damaged. Therefore, laser treatment itself becomes impossible.

Further, this type of laser treatment requires multiple irradiations of the laser light. Thereby, it is concerned that the damage to the tissue in site other than the irradiated target site may be further increased.

Further, when the irradiated target site is away from an optical axis of an irradiation optical system for irradiating the laser light, the influence of aberration increases. It becomes even more difficult to irradiate the irradiated target site with the laser light or aiming light.

According to some embodiments of the present invention, a laser treatment apparatus capable of reliably irradiating a desired site with laser light, and a method for controlling the laser treatment apparatus can be provided.

Referring now to the drawings, exemplary embodiments of an laser treatment apparatus and a method of controlling the laser treatment apparatus according to the present invention are described below. In the embodiments, any of the techniques disclosed in the documents cited in the present specification can be applied to the embodiments below.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

In the following embodiments, a direction from the apparatus optical system mounted on the laser treatment apparatus toward the patient is referred to as a “Z direction”. Further, a horizontal direction orthogonal to the Z direction is referred to as a “X direction”. Furthermore, a direction orthogonal to both the X direction and the Z direction is referred as a “Y direction”.

### [Configuration]

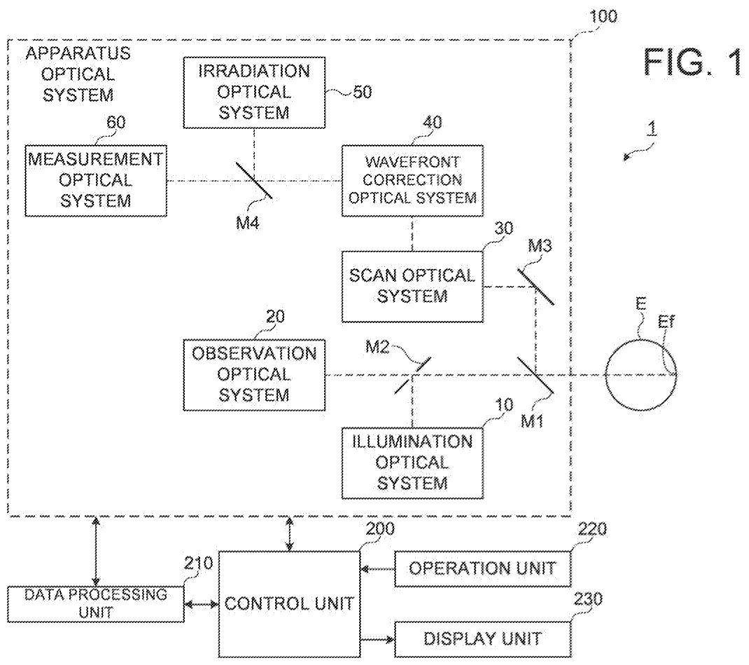

FIG. 1 shows a functional block diagram of an outline of the configuration of the laser treatment apparatus according to the embodiments. The laser treatment apparatus 1 includes an apparatus optical system 100 and a control unit (controller) 200. The apparatus optical system 100 includes an optical system for irradiating a patient's eye E with laser light (laser beam). The control unit 200 controls each part of the laser treatment apparatus 1.

The laser treatment apparatus 1 may be provided with a data processing unit (data processor) 210, an operation unit 220, and a display unit 230. The control unit 200 controls the data processing unit 210, the operation unit 220, and the display unit 230. Further, the laser treatment apparatus 1 may be further provided with an image forming unit (image former) described later. In this case, the control unit 200 can control the image forming unit.

The apparatus optical system 100 includes an illumination optical system 10, an observation optical system 20, a scan optical system 30, a wavefront correction optical system 40, an irradiation optical system 50, and a measurement optical system 60. An observation system of the apparatus optical system 100 includes, for example, the illumination optical system 10 and the observation optical system 20.

The apparatus optical system 100 includes an optical element as an optical path coupling/separating member for separating an optical path of the optical system described above or coupling with another optical system. In FIG. 1, for example, a beam splitter M1, a perforated mirror M2, a reflective mirror M3, and a beam splitter M4 are provided as the optical path coupling/separating members.

The beam splitter M1 couples an optical path of the observation system and an optical path of the other optical system, or separates an optical path of returning light from the patient's eye E into the optical path of the observation system and the optical path of the other optical system. The beam splitter M1 has characteristics of transmitting light from the observation system, and of reflecting light from the other optical system (the measurement optical system 60 or the irradiation optical system 50). It is preferred that the beam splitter M1 couples the optical paths of the observation optical system and the other optical system so that an optical axis of the observation system is substantially coaxial with an optical axis of the other optical system.

### (Observation System)

The observation system of the apparatus optical system 100 includes the illumination optical system 10 and the observation optical system 20, described above. The illumination optical system 10 illuminates a fundus Ef of the patient's eye E. The illumination optical system 10 includes an illumination light source, a lens, and the like. The observation optical system 20 is used for observing the fundus Ef illuminated by the illumination optical system 10.

The perforated mirror M2 couples an optical path of the illumination optical system 10 with an optical path of the observation optical system 20. It is preferred that the perforated mirrors M2 couples both of the optical paths of the illumination optical system 10 and the observation optical system 20 so that the optical axis of the illumination optical system 10 is substantially coaxial with the optical axis of the observation optical system 20. A hole part formed in the perforated mirror M2 is, for example, disposed at a position optically substantially conjugate with a pupil of the patient's eye E, as described later. Fundus illumination light from the illumination optical system 10 is reflected on a peripheral part of the hole part formed in the perforated mirror M2, and is guided to the fundus Ef of the patient's eye E. Returning light of the fundus illumination light from the fundus Ef passes through the hole part formed in the perforated mirror M2, and is guided to the observation optical system 20. The observation optical system 20 includes at least one of an eyepiece and an imaging element. The eyepiece is used for observing the fundus Ef with the naked eye(s). The imaging element is used for acquiring a front image of the fundus Ef. The control unit 200 that has received a signal from the imaging element controls the display unit 230 to display the image acquired using the imaging element on a display (not shown) or the like.

### (Measurement Optical System)

The measurement optical system 60 is an optical system configured to project light onto the patient's eye E and to receive returning light from the patient's eye E. Such the measurement optical system 60 includes at least one of a wavefront measurement optical system and an interference optical system. The wavefront measurement optical system is used for receiving returning light from the patient's eye E and measuring a wavefront aberration of the received returning light. The interference optical system is configured to split light from a light source into measurement light and reference light, to irradiate the patient's eye E with the measurement light, and to guide interference light to a detection means. Here, the interference light is obtained by superimposing returning light from the patient's eye E (fundus Ef) and the reference light. In this case, in the laser treatment apparatus 1, for example, a swept source type or a spectral domain type OCT is applied.

The control unit 200 can control the wavefront correction optical system 40 based on a light receiving result of the returning light from the patient's eye E obtained by the measurement optical system 60. For example, the control unit 200 controls the wavefront correction optical system 40 based on a irradiated target position. Thereby, the focal position of the laser light (laser light for treatment (therapeutic laser light)) can be arranged in the vicinity of a desired irradiated position. Further, for example, the control unit 200 controls the wavefront correction optical system 40 based on a calculation result of the wavefront aberration obtained by the wavefront measurement optical system. Thereby, the wavefront correction optical system 40 can be controlled in accordance with the shape of the fundus Ef.

Further, the control unit 200 can control the wavefront correction optical system 40 based on a detection result of the interference light obtained by the interference optical system. In this case, the control unit 200 controls the wavefront correction optical system 40 so that a designated position in a tomographic image formed based on the detection result of the interference light matches the irradiated target position of the laser light. Further, the control unit 200 can control the wavefront correction optical system 40 based on an intensity of the interference light (interference signal) specified based on the detection result of the interference light.

### (Irradiation Optical System)

The irradiation optical system 50 irradiates the fundus Ef with the laser light for treatment. The laser light for treatment is used for laser therapy (photocoagulation, photoablation, etc.) of the fundus Ef. The irradiation optical system 50 may be provided with a laser light source for treatment that outputs the laser light for treatment. The laser light for treatment may be visible laser light or invisible laser light according to its usage. The laser light source for treatment is controlled by the control unit 200.

The irradiation optical system 50 may have a function for irradiating the fundus Ef with an aiming light for aiming the laser light for treatment (irradiated light). The irradiation optical system 50 may be provided with an aiming light source that outputs the aiming light. For example, in the case of applying a configuration that performs aiming while observing the fundus Ef with the naked eye(s), a light source emitting visible light recognizable by an operator's eye (laser light source, light emitting diode, etc.) is used as the aiming light source. Alternatively, in the case of applying a configuration that performs aiming while observing a photographic image of the fundus Ef, a light source emitting light in a wavelength band in which the image sensor for acquiring the photographic image has sensitivity (laser light source, light emitting diode, etc.) is used as the aiming light source The operation of the aiming light source is controlled by the control unit 200.

For example, the laser light for treatment (or aiming light) output from the irradiation optical system 50 is irradiated to the fundus Ef in accordance with a predetermined irradiation pattern by the scan optical system 30 (described later) controlled by the control unit 200. There are various conditions (irradiation conditions) for the irradiation pattern of the laser light for treatment. A projection image of the laser light for treatment (that is, irradiated range of the laser light for treatment on the fundus Ef) is referred to as a spot. Examples of the irradiation condition include arrangement of spots (arrangement condition), size of arrangement (arrangement size condition), orientation of arrangement (arrangement orientation condition), size of each spot (spot size condition), intervals between spots (spot interval condition), number of spots (spot number condition), etc.

The arrangement condition indicates how a plurality of spots is arranged. As disclosed in Japanese Unexamined Patent Application Publication No. 2015-058152, examples of the arrangement condition include a circular arrangement, an elliptic arrangement, a rectangular arrangement, an arc-shaped arrangement, a linear arrangement, a disc-shaped arrangement, an elliptic-plate-shaped arrangement, a rectangular-plate-shaped arrangement, a fan-plate-shaped arrangement, a circular arrangement with width (annulus-ring-shaped arrangement), an arc-shaped arrangement with width (a part of annulus-ring-shaped arrangement: partial annulus-ring-shaped arrangement), and a linear arrangement with width (strip-shaped arrangement). Further, a user may set the arrangement arbitrarily. Moreover, a combination of two or more arrangements may be applied. The arrangement conditions are used for controlling the scan optical system 30. Further, the arrangement conditions may be used for controlling the wavefront correction optical system 40.

The beam splitter M4 couples an optical path of the measurement optical system 60 and the optical path of the irradiation optical system 50, or separates the optical path of the measurement optical system 60 from the optical path of the returning light from the patient's eye E. It is preferred that the beam splitter M4 couples both of the optical paths of the measurement optical system 60 and the irradiation optical system 50 so that the optical axis of the measurement optical system 60 is substantially coaxial with the optical axis of the irradiation optical system 50. Light from the measurement optical system 60 is transmitted through the beam splitter M4, an is guided to the patient's eye E through the wavefront correction optical system 40 and the scan optical system 30. The light from the irradiation optical system 50 (laser light for treatment or aiming light) is reflected by (on) the beam splitter M4, and is guided to the patient's eye E through the wavefront correction optical system 40 and the scan optical system 30. Returning light (from the patient's eye E) of the light from the measurement optical system 60 is transmitted through the beam splitter M4, and is received in the measurement optical system 60. Returning light (from the patient's eye E) of the aiming light from the irradiation optical system 50 is transmitted through the beam splitter M1, and enters the observation optical system 20.

### (Wavefront Correction Optical System)

The wavefront correction optical system 40 changes at least the wave front of the light from the irradiation optical system 50, and guides the light whose wave front is changed to the patient's eye E. Such the wavefront correcting optical system 40 includes a deformable mirror. The wavefront correction optical system 40 can change at least the wave front of the light from the irradiation optical system 50, under the control of the control unit 200. Thereby, the focal position of the light from irradiation optical system 50 in the patient's eye E can be changed at least in the Z direction. The wavefront correction optical system 40 is an example of a wavefront operation optical system that operates the wave front, a wavefront change optical system that changes the wave front, or a wavefront control optical system that controls the wave front. It should be noted that the wavefront correction optical system 40 may change the wave front of the light from the measurement optical system 60, and may guide the light whose wave front is changed to the patient's eye E.

### (Scan Optical System)

The scan optical system 30 deflects the light whose wave front is corrected by the wavefront correction optical system 40, and guides the deflected light to the patient's eye E. Such the scan optical system 30 includes an optical scanner such as a galvano mirror. The scan optical system 30 can deflect the light whose wave front is corrected by the wavefront correction optical system 40, under the control of the control unit 200. Thereby, the irradiated position of the light from irradiation optical system 50 in the patient's eye E can be changed in at least one of the X direction and the Y direction.

The light deflected by the scan optical system 30 is reflected by reflective mirror M3, and is guided to the beam splitter M1. The light guided to the beam splitter M1 is reflected toward the patient's eye E by the beam splitter M1.

The control unit 200 includes a controller and a storage unit. The functions of the controller are implemented by a processor, for example. In this specification, the function of the processor is implemented by a circuit(s) such as, for example, a CPU (central processing unit), a GPU (graphics processing unit), an ASIC (application specific integrated circuit), and a PLD (programmable logic device). Examples of PLD include a simple programmable logic device (SPLD), a complex programmable logic device (CPLD), and a field programmable gate array (FPGA). The storage unit stores, in advance, a computer program for controlling the laser treatment apparatus 1. The computer program includes, for example, various light source control programs, wavefront correction control program, scan optical system control program, various detector control programs, image forming program, data processing program, program for user interface, and the like. The control unit 200 operates according to the computer programs, and thereby the control unit 200 performs the control process.

The data processing unit 210 performs various types of data processing (image processing) and/or analysis processing on the light receiving result obtained using the apparatus optical system 100, under the control of the control unit 200. For example, the data processing unit 210 performs various correction processes such as brightness correction and dispersion correction of images. Further, the data processing unit 210 performs various kinds of image processing and various kinds of analysis processing on fundus images, anterior segment images, or tomographic images. The data processing unit 210 can form volume data (voxel data) of the patient's eye E by performing known image processing such as interpolation processing for interpolating pixels between tomographic images. In the case of displaying an image based on the volume data, the data processing unit 210 performs rendering processing on the volume data so as to form a pseudo three-dimensional image viewed from a specific line-of-sight direction.

The data processing unit 210 includes a controller and a storage unit, similar to the control unit 200. The controller operates in accordance with the computer program stored in advance in the storage unit. Thereby, the data processing unit 210 executes data processing.

The operation unit 220 is used by the user to input instructions to the laser treatment apparatus 1. The operation unit 220 may include a known operation device used for a computer. For example, the operation unit 220 may include a pointing device such as a mouse, a touch pad or a track ball. Further, the operation unit 220 may include a keyboard, a pen tablet, a dedicated operation panel, or the like.

The display unit 230 includes a display such as a liquid crystal display. The display unit 230 displays various information such an image, under the control of the control unit 200. Note that the display unit 230 and the operation unit 220 need not necessarily be formed as separate unit. For example, a device like a touch panel, which has a display function integrated with an operation function, can be used.

The laser treatment apparatus 1 is provided with an optical system movement unit (not shown) for three-dimensionally moving the apparatus optical system 100. Thereby, the patient's eye E and the apparatus optical system 100 can be relatively moved. The optical system movement unit may move only a part of the optical system in the apparatus optical system 100 shown in FIG. 1. The optical system movement unit is provided with a holding member that holds the optical system to be moved (for example, the apparatus optical system 100), an actuator that generates a driving force for moving the holding member, and a transmission mechanism that transmits the driving force from the actuator to the holding member. The actuator includes a pulse motor, for example. The transmission mechanism includes a combination of gears, a rack and pinion, and the like, for example. The control unit 200 is capable of controlling the optical system movement unit to three-dimensionally move the optical system installed in the apparatus optical system 100. For example, this control is used for alignment and tracking. Here, the tracking is to move the apparatus optical system 100 according to the movement of the patient's eye E. To perform tracking, alignment and focusing are performed in advance. The tracking is performed by moving the optical system of the device in real time according to the position and orientation of the patient's eye E based on the moving image obtained by imaging the patient's eye E, thereby maintaining a suitable positional relationship in which alignment and focusing are adjusted.

### Operation Example

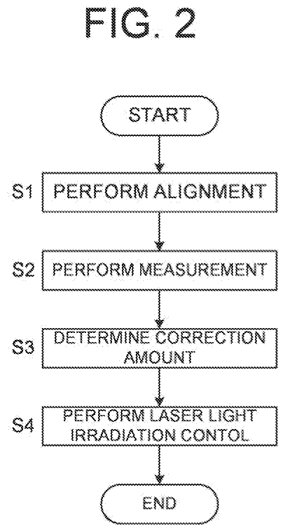

FIG. 2 shows an example of the operation of the laser treatment apparatus 1 according to the embodiments. The storage unit of the control unit 200 stores a of computer programs for realizing the processing shown in FIG. 2. The controller of the control unit 200 operates according to the computer programs, and thereby the control unit 200 performs the processing shown in FIG. 2.

### (S1)

First, the control unit 200 controls the optical system movement unit to move the apparatus optical system 100 to an initial position. After then, the control unit 200 performs alignment for position matching of the apparatus optical system 100 with respect to the patient's eye E.

The control unit 200 can cause the display of the display unit 230 to display the fundus image of the patient's eye E (front image of the fundus Ef) acquired using the observation system, and can control the optical system movement unit so as to move the apparatus optical system 100 in a designated direction by the user using the operation unit 220. In this case, the observation optical system 20 acquires the front image of the patient's eye E illuminated by the illumination optical system 10.

Alternatively, the control unit 200 may cause the display of the display unit 230 to display the anterior segment image of the patient's eye E acquired using an anterior segment imaging system (not shown), and can control the optical system movement unit so as to move the apparatus optical system 100 in a designated direction by the user using the operation unit 220.

Further, the control unit 200 may perform position matching of the apparatus optical system 100 with respect to the patient's eye E, by projecting light from an alignment light source (not shown) onto the patient's eye E to control the optical system movement unit based on an image corresponding to returning light of the light.

Alternatively, the control unit 200 may perform position matching of the apparatus optical system 100 with respect to the patient's eye E, by photographing the anterior segment of the patient's eye E from different directions each other using two or more cameras (not shown), specifying a position of the patient's eye E from two or more images with parallax, and controlling the optical system movement unit based on the specified position of the patient's eye E.

The control unit 200 can perform focus adjustment and start tracking after the alignment is completed.

The control unit 200 can specify an in-focus state (degree of blur) of the fundus image acquired by observing system, and can perform focus adjustment by moving the apparatus optical system 100 etc. so that the specified in-focus state becomes a desired in-focus state. Alternatively, the control unit 200 may photograph the anterior segment from different directions each other using the two or more cameras, may specify the in-focus stat from the two or more images with parallax, and may obtain a movement amount in the Z direction of the apparatus optical system 100 so that the specified in-focus state becomes a desired in-focus state.

Further, the control unit 200 can repeatedly obtain the image of the patient's eye E using the observation system, and can specify a characteristic site in the image acquired at a predetermined timing, and can perform tracking by controlling the optical system movement unit so as to cancel a displacement amount when the position of the characteristic site is changed.

### (S2)

Next, the control unit 200 control the measurement optical system 60 to start measuring the patient's eye E. The measurement optical system 60 projects light onto the patient's eye E and detects returning light from the patient's eye E. In case that the measurement optical system 60 includes the wavefront measurement optical system, the measurement optical system 60 detects, for example, a great number of light fluxes, which are obtained by dividing the returning light, using the Shack-Hartmann sensor. In case that the measurement optical system 60 includes the interference optical system, the measurement optical system 60 detects, for example, the interference light obtained by interfering the returning light and the reference light.

### (S3)

Subsequently, the control unit 200 causes the data processing unit 210 to calculate a correction amount (wavefront aberration correction amount, control amount) for the wavefront correction optical system 40 based on the light receiving result of the returning light obtained in step S2.

The data processing unit 210 can calculate the correction amount based on the predetermined irradiated target position of the laser light for treatment and the light receiving result of the returning light obtained in step S2. For example, the correction amount for the Z position (a position in the Z direction with respect to the reference position) of the irradiated target position is calculated from the obtained light receiving result of the returning light. The irradiated target position may be a predetermined irradiated target position, or an irradiated target position designated using the operation unit 220.

The content of the data processing performed by the data processing unit 210 corresponds to a measurement result obtained by the measurement optical system 60. In the case that the measurement optical system 60 includes the wavefront measurement optical system, the data processing unit 210 calculates the wavefront aberration using a known method based on the measurement result obtained by the measurement optical system 60, for example. Further, the data processing unit 210 obtains the correction amount for the wavefront correction optical system 40 from the calculated wavefront aberration so that the focal position of the laser light matches the Z position of the irradiated target position. The focal position of the laser light is specified from, for example, the positional relationship between the apparatus optical system 100 and the patient's eye E and the beam shape of the laser light measured in advance. Alternatively, the focal position of the laser light may be specified by performing known processing such as a ray trace processing.

In the case that the measurement optical system 60 includes the interference optical system, the data processing unit 210 or the image forming unit 240 described later forms the tomographic image based on the measurement result obtained by the measurement optical system 60. The data processing unit 210 obtains the correction amount for the wavefront correction optical system 40 corresponding to the irradiated target position previously determined in the formed tomographic image or the irradiated target position designated in the tomographic image using the operation unit 220. In this case, the data processing unit 210 can store table information in which the correction amount for the wavefront correction optical system 40 is set in advance corresponding to the irradiated target position in the Z direction in the tomographic image, and can obtain the correction amount based on the table information.

Alternatively, in the case that the measurement optical system 60 includes the interference optical system, the data processing unit 210 may calculate the intensity of the interference signal (detection result) obtained by detecting the interference light, based on the measurement result obtained by the measurement optical system 60, for example. The data processing unit 210 can calculate the intensity of the interference signal (intensity of the interference light) by performing known processing on the detection result of the interference light. Further, the data processing unit 210 may calculate the correction amount for the wavefront correction optical system 40 from the calculated intensity of the interference signal. In this case, the data processing unit 210 can store table information in which the correction amount for the wavefront correction optical system 40 is set in advance corresponding to the intensity of the interference signal, and can obtain the correction amount based on the table information.

### (S4)

The control unit 200 performs laser light irradiation control. The laser light irradiation control includes laser output control, wavefront correction control, and scan control. The control unit 200 performs laser light irradiation control for outputting the laser light under a predetermined output condition, by controlling a laser irradiation light source included in the irradiation optical system 50. The control unit 200 performs wavefront correction control for correcting the wave front using the wavefront correction optical system 40 on the laser light output from the irradiation optical system 50, based on the correction amount obtained in step S3. The control unit 200 performs scan control for controlling the deflecting direction of the laser light deflected by the scan optical system 30 so that the laser light whose wave front is corrected by the wavefront correction optical system 40 irradiates the irradiated target position set in advance. The position in the X direction and the position in the Y direction of the irradiated target position are changed by deflection control performed by the scan optical system 30. The position in the Z direction of the irradiated target position is changed by the wavefront correction control performed by the wavefront correction optical system 40. This terminates the operation of the laser treatment apparatus 1 (END).

It should be noted that step S2 and step S3 may be omitted, and in step S4, the control unit 200 may control the wavefront correction optical system 40 using a predetermined correction amount.

Alternatively, in step S3, the data processing unit 210 may repeatedly acquire the light receiving result of the returning light by the measurement optical system 60, and may calculate the correction amount for the wavefront correction optical system 40 so that the intensity of the interference signal is maximized.

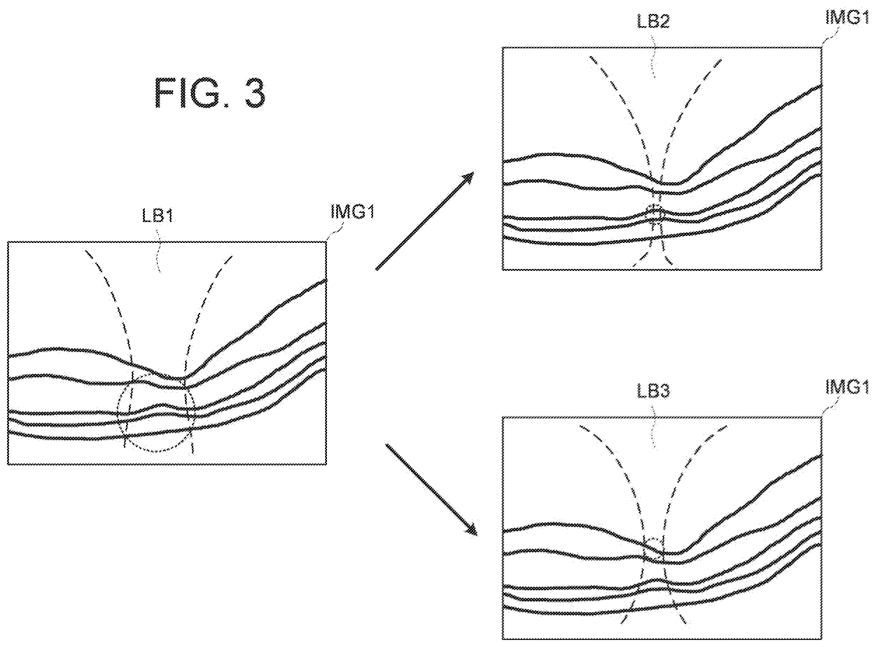

FIG. 3 shows a diagram describing the operation of the laser treatment apparatus 1 according to the embodiments. FIG. 3 schematically shows the cross-sectional shape of the beam of the laser light irradiated to the fundus Ef of the patient's eye E.

For example, the control unit 200 can superimpose a predicted image LB1 on the tomographic image IMG1 and display it on the display of the display unit 230, based on the measurement result acquired by the measurement optical system 60 obtained in step S2 of FIG. 2. Here, the predicted image LB1 is an image of the cross-sectional shape in the Z direction of the beam of the laser light. The predicted image LB1 is obtained by performing correction corresponding to the measurement result by the measurement optical system 60 on the cross-sectional shape of the beam of the laser light measured in advance. Alternatively, the predicted image LB1 of the cross-sectional shape of the beam of the laser light may be generated by known processing such as ray trace processing.

As described above, the control unit 200 causes the data processing unit 210 to calculate the correction amount so that the focal position of the laser light matches the predetermined irradiated target position, and controls the wavefront correction optical system 40 based on the calculated correction amount. Thereby, the cross-sectional shape in the Z direction of the beam can be reduced without moving the focal position of the laser light (beam LB2). Therefore, the irradiation of the laser light to the site around the irradiated target position can be avoided.

Further, the control unit 200 can control the wavefront correction optical system 40 based on the calculated correction amount to move the focal position of the laser light in the Z direction (beam LB3).

It should be noted that the control unit 200 can control the scan optical system 30 based on the irradiated target position set in advance to move the irradiated position of the laser light in the fundus Ef in at least one of the X direction and the Y direction. Alternatively, the control unit 200 may control the wavefront correction optical system 40 based on the irradiated target position set in advance to move the irradiated position of the laser light in the fundus Ef at least one of the X direction and the Y direction. Alternatively, the control unit 200 may control the wavefront correction optical system 40 and the optical system movement unit (not shown) to move the irradiated position of the laser light in the Z direction. Alternatively, the control unit 200 may control the optical system movement unit (not shown) alone to move the irradiated position of the laser light in the Z direction.

Hereinafter, a specific configuration example of the laser treatment apparatus 1 according to the embodiments will be described.

### First Configuration Example

In a first configuration example, in the apparatus optical system 100, the measurement optical system 60 includes a wavefront measurement optical system 60A, and an interference optical system 70 is separately provided for confirming the laser treatment site. The interference optical system 70 is mainly used for acquiring a tomographic image of a site irradiated with laser light. It should be noted that the interference optical system 70 may be omitted in the first configuration example.

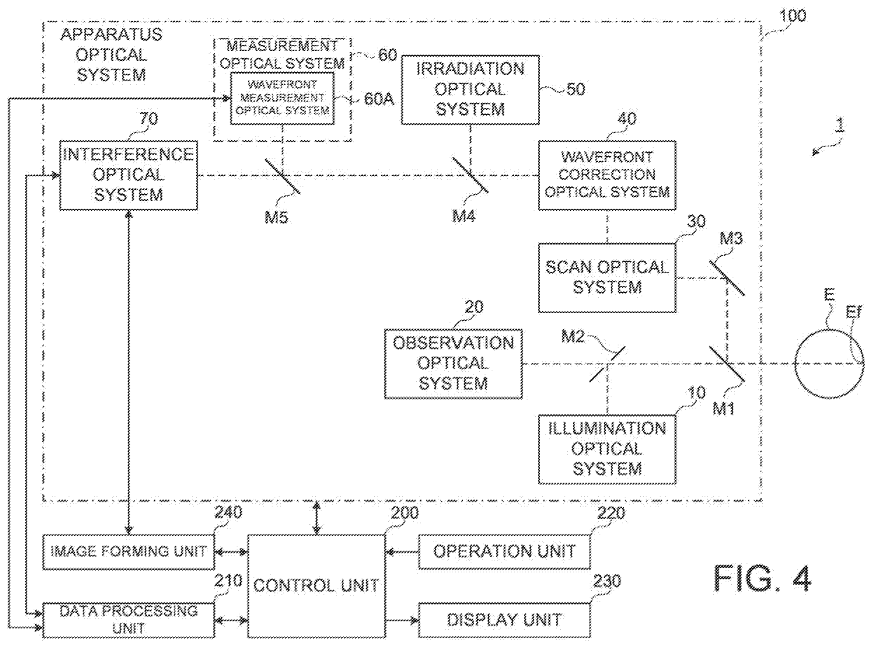

FIG. 4 shows a functional block diagram of the configuration of the laser treatment apparatus according to the first configuration example of the embodiments. In FIG. 4, parts similarly configured to those in FIG. 1 are denoted by the same reference numerals, and the description thereof is omitted unless it is necessary.

In the apparatus optical system 100 according to the first configuration example, the interference optical system 70 and a beam splitter M5 are added to the apparatus optical system shown in FIG. 1. The beam splitter M5 couples an optical path of the interference optical system 70 and an optical path of the wavefront measurement optical system 60A (measurement optical system 60), or separates the optical path of the interference optical system 70 (or the measurement optical system 60) from the optical path of the returning light from the patient's eye E. It is preferred that the beam splitter M5 couples both of the measurement optical system 60 and the interference optical system 70 so that the optical axis of the measurement optical system 60 is substantially coaxial with the optical axis of the interference optical system 70.

For example, light from the interference optical system 70 is transmitted through the beam splitter M4, and is guided to the patient's eye E through the wavefront correction optical system 40 and the scan optical system 30. Returning light (from the patient's eye E) of the light from the interference optical system 70 is transmitted through the beam splitter M4, is transmitted through the beam splitter M5, and is used for generating the interference light in the interference optical system 70.

For example, light from the measurement optical system 60 is reflected by the beam splitter M5, is transmitted through the beam splitter M4, and is guided to the patient's eye E through the wavefront correction optical system 40 and the scan optical system 30. Returning light (from the patient's eye E) of the light from the measurement optical system 60 is transmitted through the beam splitter M4, is reflected by the beam splitter M5, and is received in the measurement optical system 60.

The laser treatment apparatus 1 according to the first configuration example is provided with the image forming unit 240. The image forming unit (image former) 240 forms various types of images (image data). The image forming unit 240 forms a tomographic image (OCT image) of the patient's eye E based on the detection result of the interference light obtained by the interference optical system 70. Specifically, the image forming unit 240 forms image data of the tomographic image of the fundus Ef of the patient's eye E based on the detection result of the interference light obtained by the interference optical system 70 and a pixel position signal input from the control unit 200. The image forming unit 240 can apply Fourier transform and the like to the spectral distribution based on the detection result of the interference light, for example, every series of wavelength scans (every A-line) to form the reflection intensity profile in each A-line. The image forming unit 240 can form image data by imaging the reflection intensity profile in each A-line.

Further, the image forming unit 240 can form an anterior segment image of the patient's eye E based on the detection result of the returning light from the anterior segment of the patient's eye E obtained by the imaging element in the observation optical system 20.

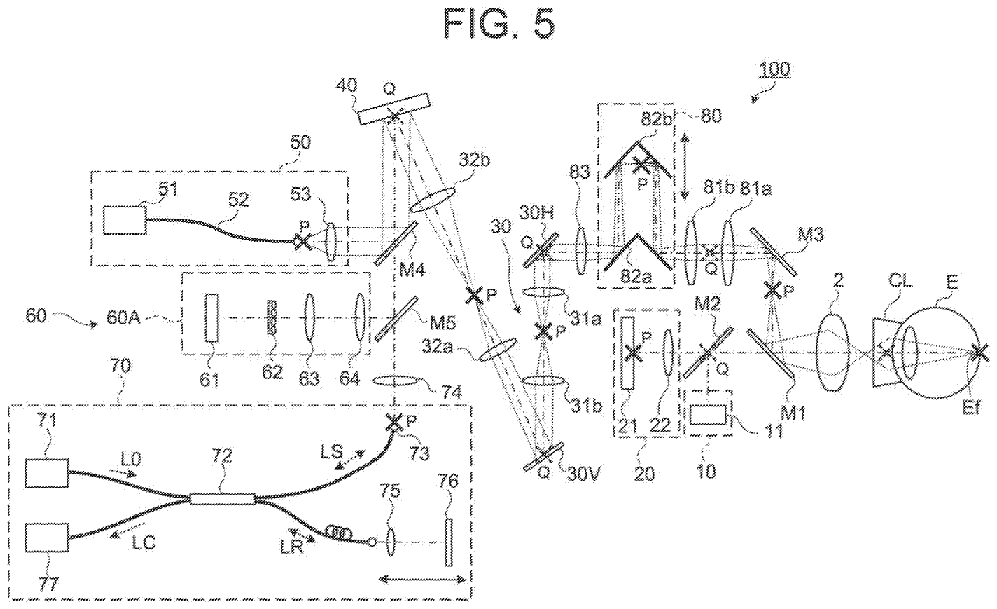

FIG. 5 shows a schematic diagram of the configuration of the optical system of the laser treatment apparatus 1 according to the first configuration example of the embodiments. In FIG. 5, a contact lens CL used for laser treatment of the fundus Ef is shown. When the laser treatment is not performed, the contact lens CL may be capable of being removed from the optical axis of the apparatus optical system 100. In FIG. 5, parts similar to those in FIG. 4 are denoted by the same reference symbols, and description thereof is omitted as appropriate. In FIG. 5, a position optically conjugate with the fundus Ef of patient's eye E is illustrated as a fundus conjugate position P, and a position optically conjugate with the pupil of the patient's eye E is illustrated as a pupil conjugate position (anterior segment conjugate position) Q.

The apparatus optical system 100 is provided with an objective lens 2. The objective lens 2 is arranged at a position facing the patient's eye E. The objective lens 2 may have a structure in which a plurality of lenses is combined in order to suppress aberration. Alternatively, the objective lens 2 may be formed of a single lens. As shown in FIG. 5, the objective lens 2 is disposed between the beam splitter M1 and the contact lens CL (patient's eye E), during laser irradiation of the fundus Ef

### (Observation System)

The illumination optical system 10 includes an illumination light source 11 that outputs illumination light for illuminating the fundus Ef of the patient's eye E. The illumination light source 11 includes a halogen lamp or a light emitting diode (LED). The illumination optical system 10 may be provided with a lens that refracts light output from the illumination light source 11. The operation of the illumination light source 11 is controlled by the control unit 200.

A hole part (perforated part) formed in the perforated mirror M2 is arranged at the pupil conjugate position Q or near the pupil conjugate position Q.

The observation optical system 20 includes an imaging element 21 and an imaging lens 22. The detection surface of the imaging element 21 is arranged at the fundus conjugate position P or near the fundus conjugate position P. The imaging lens 22 is arranged between the imaging element 21 and the perforated mirror M2.

Illumination light output from the illumination light source 11 is reflected on a peripheral region of the hole part of the perforated mirror M2, passed through the beam splitter M1, the objective lens 2, and the contact lens CL, and illuminates the fundus Ef of the patient's eye E. Returning light of the illuminating light from the fundus Ef travels through the same path in the opposite direction, and passes through the hole part of the perforated mirror M2. The returning light passing through the hole part of the perforated mirror M2 is condensed on the detection surface of the imaging element 21 by the imaging lens 22. The imaging element 21 includes a charge-coupled device (CCD) or a complementary metal-oxide semiconductor (CMOS) image sensor, for example. The detection result of the returning light from the patient's eye E obtained by the imaging element 21 is used for forming an fundus image.

### (Irradiation Optical System)

The irradiation optical system 50 includes a light source 51, an optical fiber 52, and a lens 53. The light source 51 emits laser light (laser light for treatment) having a wavelength component(s) (for example, 638 nm) selected from the wavelength range of 500 nm to 900 nm, for example. Examples of the light source 51 include a laser diode (LD), a super luminescent diode (SLD), a laser driven light source (LDLS), and the like. The laser light emitted from the light source 51 is not limited to light of a single wavelength, and may have wavelength components of a certain band width. The light source 51 may emit light having high directivity (i.e., light with small divergence angle).

The light source 51 is connected to the optical fiber 52. The optical fiber 52 guides the laser light to the lens 53. The optical fiber 52 is a single mode fiber. The core diameter of the optical fiber 52 may be substantially equal to the spot diameter of the laser light emitted from the light source 51. The lens 53 is located at the exit end of the optical fiber 52. The lens 53 collimates the laser light output from the optical fiber 52 to generate a parallel light flux. The laser light having passed through the lens 53 are guided to a beam splitter M4.

### (Wavefront Measurement Optical System)

Reflected light of light for measuring wavefront aberration from the fundus Ef is incident on the wavefront measurement optical system 60A. The light is output from a light source included in an optical system separately provided such as the irradiation optical system 50 and the interference optical system 70. It should be noted that the wavefront measurement optical system 60A may be provided with an optical system for irradiating the fundus Ef of the patient's eye E with the light for measuring wavefront aberration, and that the reflected light of the light for measuring the wavefront aberration from the fundus Ef may be configured to enter a lens array 62. In this case, the light for measurement wavefront aberration output from the wavefront measurement optical system 60A is reflected toward the fundus Ef by the beam splitter M5. The reflected light from the fundus Ef is reflected by the beam splitter M5, and is guided to a CCD 61 described later.

The wavefront measurement optical system 60A includes a Shack-Hartmann sensor. Specifically, the wavefront measurement optical system 60A includes the CCD 61 as the imaging element, the lens array 62 arranged in front of the CCD 61, and lenses 63 and 64. The lens array 62 is formed of small lenses which are arranged in a grid pattern. The lens array 62 divides the incident light into a number of light fluxes, and condenses each of the light fluxes. The focal points of the lens array 62 are captured by the CCD 61. By analyzing the focal positions of the lenses, the wavefront aberration of the light incident on the lens array 62 can be detected. That is, by observing a reflection image from the fundus Ef of the patient's eye E through the lens array 62, the disturbance of a wave front in the reflection image can be detected. The image captured by the CCD 61 is sent to an image analyzer such as the control unit 200, the image forming unit 240, or the data processing unit 210. The image analyzer analyzes the disturbance of the wave front. Thus, a control signal (feedback signal), which is based on the result of the analysis, is sent to the wavefront correction optical system 40.

### (Interference Optical System)

The interference optical system 70 is provided with an optical system for acquiring a tomographic image of a measurement site such as the fundus Ef. This optical system has the same configuration as the conventional Fourier domain OCT device. That is, this optical system is configured to: split light (low coherence light) from the light source unit (OCT light source) into reference light LR and measurement light LS; superpose returning light of the measurement light LS, which has traveled through the fundus Ef, on the reference light LR, which has traveled through the reference light path, to generate interference light LC; and detect the spectral components of the interference light LC. The detection result (detection signal) is sent to the image forming unit 240.

The light source unit 71 includes a wavelength sweeping type (i.e., a wavelength scanning type) light source capable of sweeping (scanning) the wavelengths of the emitted light. For example, a laser light source, which includes a resonator and emits light having a predetermined center wavelength, is used as the wavelength sweeping type light source. The light source unit 71 temporally changes the output wavelength in the near infrared wavelength band that cannot be visually recognized by human eyes.

Light L0 output from the light source unit 71 may be, for example, near-infrared light including a wavelength band different from the laser light emitted from the light source 51. For example, the light L0 may be near infrared light having a center wavelength of about 1040 nm to 1060 nm (for example, 1050 nm) and a wavelength width of about 50 nm. In the embodiments, the swept source type is particularly described. However, when the spectral domain type OCT is applied, a light output device, such as a super luminescent diode (SLD), an LED, a semiconductor optical amplifier (SOA), or the like is used as the light source unit 71. Generally, the configuration of the light source unit 71 is selected as appropriate according to the type of optical coherence tomography.

The light L0 output from the light source unit 71 is guided to the fiber coupler 72 through an optical fiber, and is split into the measurement light LS and the reference light LR.

The measurement light LS is guided through an optical fiber, and is emitted from the fiber end 73. The measurement light LS emitted from the fiber end 73 is collimated into a parallel light flux by a collimator lens 74. This fiber end 73 of the optical fiber is arranged at the fundus conjugate position P or near the fundus conjugate position P. An optical path of the measurement light LS is coupled with an optical path of the wavefront measurement optical system 60A (measurement optical system 60) described above. Having traveled through an optical path described later, the measurement light LS is irradiated onto the measurement sit such as the fundus Ef. The measurement light LS is scattered and reflected at, for example, the measurement site such as the fundus Ef. The scattered and reflected light may be sometimes referred to as returning light of the measurement light LS. The returning light of the measurement light LS travels through the same path in the opposite direction, and is thereby guided to the fiber coupler 72.

The reference light LR is guided through an optical fiber, is emitted from an fiber end via a polarization adjuster (polarization controller), and is collimated into a parallel light flux by a lens 75. For example, the polarization adjuster applies external stress to the looped optical fiber to thereby adjust the polarization condition of the reference light LR guided through the optical fiber. The reference light LR converted into a parallel light flux is reflected in the opposite direction by the reference mirror 76 and is condensed again by the lens 75 at a fiber end of an optical fiber. A reference unit including a lens 75 and the reference mirror 76 is integrally movable along the traveling direction of the reference light LR. The correction according to the axial length of the eye can be performed by moving the reference unit. The reference light LR reflected on the reference mirror 76 travels through the same path in the opposite direction, and is guided to the fiber coupler 72. It should be noted that an optical element for dispersion compensation (pair prism, etc.), an optical element for polarization correction (wavelength plate, etc.), or an optical attenuator (attenuator) may be provided on the optical path of the reference light LR (reference light path). The optical attenuator adjusts the light amount of the reference light passing through the optical fiber under the control of the control unit 200.

The fiber coupler 72 superposes the returning light of the measurement light LS on the reference light LR reflected by the reference mirror 76. The interference light LC thus generated is guided to a detection unit 77 through an optical fiber. The fiber coupler 72 splits the interference light at a predetermined splitting ratio (e.g., 1:1) to generate a pair of interference light LC. The pair of interference light LC is detected by the detector (balanced photodiode) provided in the detection unit 77. It should be noted that the detector (spectrometer) detects the interference light generated by the fiber coupler by decomposing it into a plurality of wavelength components in the case of spectral domain OCT.

The detector sends a detection result (detection signal) of the pair of interference light LC to a data acquisition system (DAQ) (not shown). A clock KC is supplied from the light source unit 71 to the DAQ. This clock is generated in synchronization with the output timing of each wavelength swept within a predetermined wavelength range by the wavelength sweeping type light source. The DAQ performs sampling of the detection signal based on the clock. The sampling result is sent to the image forming unit 240 for forming an OCT image.

The wavefront correction optical system 40, the scan optical system 30, and a diopter correction optical system 80 are provided between the beam splitter M4 and the beam splitter M1.

### (Wavefront Correction Optical System)

The wavefront correction optical system 40 is disposed on the patient's eye E side of the beam splitter M4. The wavefront correction optical system 40 includes a deformable mirror. The deformable mirror is a mirror that can deform the shape of the surface by a plurality of actuators.

The deformable mirror is driven using the control signal generated by the control unit 200 so that the focal position of the laser light for treatment matches the irradiated target position.

Alternatively, the deformable mirror may be driven using the control signal based on the analysis result of an image formed using a detection result obtained by the CCD 61. For example, if a captured image based on the detection result obtained by the CCD 61 has a distortion (distortion of the wave front), the shape of the surface of the deformable mirror is deformed to reduce the distortion. That is, the shape of the surface of the deformable mirror is changed so as to reduce the distortion of the image of the fundus Ef based on the detection result obtained by the CCD 61 under the feedback control. Thus, the distortion of the image of the fundus Ef is suppressed.

The scan optical system 30 is disposed on the patient's eye E side of the wavefront correction optical system 40 through lenses 32b and 32a for adjusting light flux. The fundus conjugate position P or its vicinity is disposed between the lens 32b and the lens 32a.

### (Scan Optical System)

The scan optical system 30 is used to irradiate the fundus Ef of the patient's eye E with the laser light from the light source 51. Further, the scan optical system 30 is used to scan the fundus Ef of the patient's eye E with the measurement light LS from the interference optical system 70.

The scan optical system 30 includes a vertical optical scanner 30V and a horizontal optical scanner 30H. The vertical optical scanner 30V is a mirror whose tilt angle is variable. The tilt of the reflective surface of the mirror is controlled by the control unit 200. The vertical optical scanner 30V is used for, for example, scanning in the vertical direction in the fundus plane. The vertical optical scanner 30V may be a low-speed scanner such as a galvanometer mirror. The horizontal optical scanner 30H is located on the patient's eye E side of the vertical optical scanner 30V through the lenses 31b and 31a. The horizontal optical scanner 30H is a mirror whose tilt angle is variable. The tilt of the reflective surface of the mirror is controlled by the control unit 200. The horizontal optical scanner 30H is used for, for example, scanning in the horizontal direction perpendicular to the vertical direction in the fundus plane. Either one of the vertical optical scanner 30V and the horizontal optical scanner 30H may be a high-speed scanner such as a resonant mirror, a micro electro mechanical systems (MEMS) mirror, or the like. The reflective surface of the vertical optical scanner 30V and the reflective surface of the horizontal optical scanner 30H are arranged in the pupil conjugate positions Q or positions around there. The fundus conjugate position P or its vicinity is disposed between the lens 31a and the lens 31b.

The diopter correction optical system 80 is located on the patient's eye E side of the horizontal optical scanner 30H via a lens 83. The diopter correction optical system 80 is one example of an adjusting means for adjusting the laser light to be irradiated the fundus Ef as a substantially point image. The diopter correction optical system 80 includes diopter correction mirrors 82a and 82b. The diopter correction mirror 82a is arranged between the lens 83 and the lens 81b, and reflects incident light toward the diopter correction mirror 82b. The function of the diopter correction mirror 82b is implemented by a corner cube. The diopter correction mirror 82b reflects incident light in a direction orthogonal to the incident direction, and then emits the reflected light in a direction opposite to the incident direction.

The fundus conjugate position P or its vicinity is disposed between two reflective surfaces of the diopter correction mirror 82b. The diopter correction mirror 82b is moved in an optical axis direction by a movement mechanism (not shown). This movement mechanism is controlled by the control unit 200. The diopter correction mirror 82b continuously moves the fundus conjugate position P according to the refractive power of the patient's eye E. By relatively bringing the diopter correction mirror 82b near or far from the diopter correction mirror 82a, the focal point of the apparatus optical system 100 is adjusted to the fundus That is, there are individual differences or individual variability in diopter. Even if there is a difference in diopter, adjustment is performed so that the focal point of the apparatus optical system 100 is positioned at the fundus Ef by moving the position of the diopter correction mirror 82b. In other words, the laser light is adjusted so as to be focused and irradiated as a substantially point image on the fundus Ef Incidentally, in the diopter correction optical system 80, the pupil of the patient's eye E is in the conjugate relationship with the infinity. Therefore, the movement of the diopter correction mirror 82b does not change the pupil conjugate relationship in the apparatus optical system 100.

Reflective mirror M3 is disposed on the patient's eye E side of the diopter correction optical system 80 through the lenses 81b and 81a. The pupil conjugate position Q or its vicinity is disposed between the lens 81a and the lens 81b. The beam splitter M1 is arranged on the patient's eye E side of the reflective mirror M3. The fundus conjugate position P or its vicinity is disposed between the reflective mirror M3 and the beam splitter M1.

The irradiation optical system 50 is an example of the “irradiation system” according to the embodiments. The wavefront correction optical system 40 is an example of the “wavefront changing unit” according to the embodiments. The control unit 200 is an example of the “controller” according to the embodiments. The measurement optical system 60 is an example of the “optical system” according to the embodiments. The CCD 61 is an example of the “area sensor” according to the embodiments. The data processing unit 210 is an example of the “wavefront aberration calculator” or the “interference intensity calculator” according to the embodiments. The image forming unit 240 is an example of the “image forming unit (image former)” according to the embodiments. The operation unit 220 is an example of the “designation unit” according to the embodiments. The scan optical system 30, the vertical optical scanner 30V, and the horizontal optical scanner 30H are an example of the “optical scanner” according to the embodiments. The diopter correction optical system 80 is an example of the “diopter correction unit” according to the embodiments.

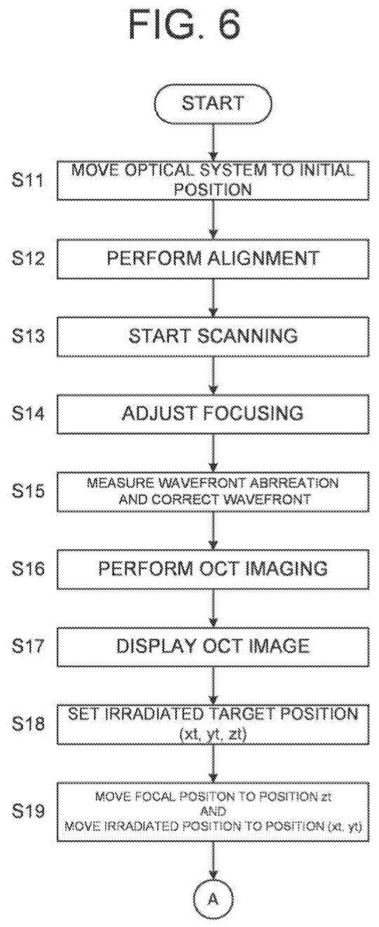

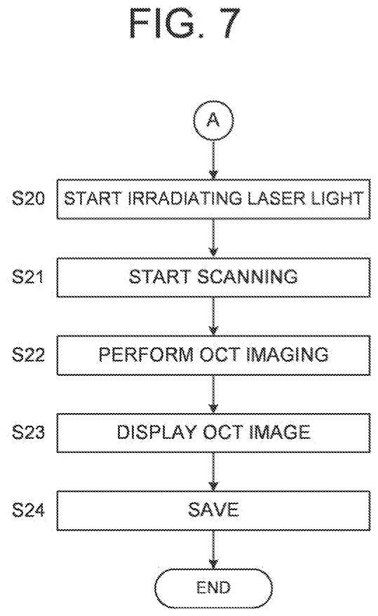

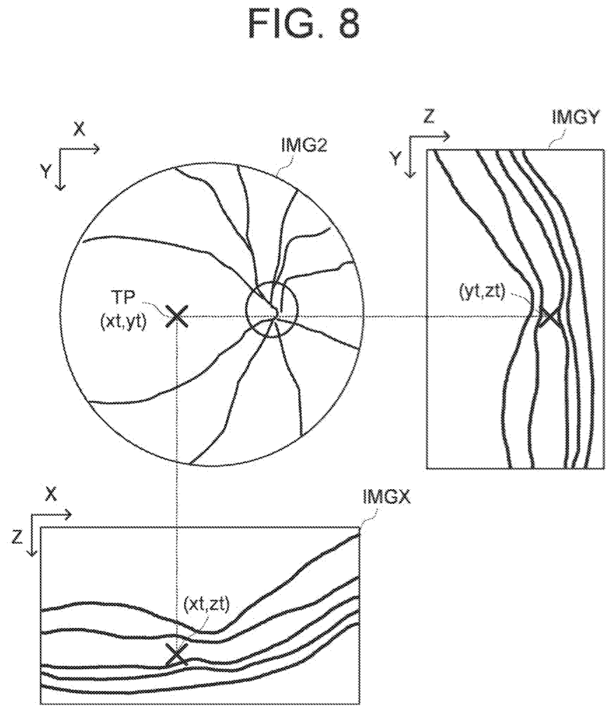

FIGS. 6 and 7 show examples of the operation of the laser treatment apparatus 1 according to the first configuration example of the embodiments. FIGS. 6 and 7 represent examples of the operation in the case where the laser light for treatment is irradiates the irradiated target position (three-dimensional position) designated using the operation unit 220 in the tomographic image of the fundus Ef as shown in FIG. 8 described later. The storage unit of the control unit 200 stores computer programs for realizing the processing shown in FIGS. 6 and 7. The controller of the control unit 200 operates according to the computer programs, and thereby the control unit 200 performs the processing shown in FIGS. 6 and 7.

### (S11)

First, the control unit 200 controls the optical system movement unit to move the apparatus optical system 100 to an initial position.

### (S12)

Subsequently, the control unit 200 performs alignment for position matching of the apparatus optical system 100 with respect to the patient's eye E, in the same manner as step S1 in FIG. 2. Further, the control unit 200 may start tracking control.

### (S13)

Next, the control unit 200 starts controlling the scan optical system 30.

### (S14)

Subsequently, the control unit 200 performs focus adjustment on the fundus Ef. For example, the control unit 200 causes the display of the display unit 230 to display the fundus image obtained by the observation system and moves the diopter correction mirror 82b by a user operation on the operation unit 220.

### (S15)

Subsequently, the control unit 200 causes the optical system to irradiate the fundus Ef with the light for measuring wavefront aberration from the light source provided in a separate optical system such as the irradiation optical system 50 or the interference optical system 70 or the measurement optical system 60. The wavefront measurement optical system 60A detects the returning light of the light for measuring wavefront aberration from the fundus Ef. The control unit 200 causes the data processing unit 210 to calculate the wavefront aberration based on the measurement result obtained by the wavefront measurement optical system 60A, and causes the data processing unit 210 to calculate the correction amount for the wavefront correction optical system 40. The control unit 200 controls the wavefront correction optical system 40 based on the calculated correction amount.

### (S16)

Next, the control unit 200 causes the interference optical system 70 to scan the fundus Ef with the measurement light LS from the interference optical system 70.

### (S17)

The control unit 200 causes the data processing unit 210 to form three-dimensional image data (volume data) of the fundus Ef based on the detection result of the interference light LC acquired by the interference optical system 70. The detection result is obtained in step S16. The control unit 200 causes the data processing unit 210 to form a tomographic image IMGX of the XZ section and a tomographic image IMGY of the YZ section from the formed three-dimensional image data, and causes the display of the display unit 230 to display them together with the fundus image IMG2 (see FIG. 8).

### (S18)

Subsequently, the control unit 200 receives the three-dimensional position (xt, yt, zt) of the irradiated target position TP of the laser light for treatment. The irradiated target position TP is designated for the fundus image IMG2, the tomographic images IMGX and IMGY displayed in step S17 by the user operation on the operation unit 220. The control unit 200 displays the designated irradiated target position TP of the laser light for treatment in an identifiable manner in the fundus image IMG2, the tomographic images IMGX and IMGY. For example, the X position and the Y position of the irradiated target position TP is a reference position of irradiation pattern representing a plurality of irradiated positions in the fundus Ef.

### (S19)

The control unit 200 controls the wavefront correction optical system 40 so that the Z position of the irradiated target position designated in step S18 becomes zt. Further, the control unit 200 controls the scan optical system 30 so that the X position of the irradiated target position designated in step S18 becomes xt and the Y position of the irradiated target position designated in step S18 becomes yt.

### (S20)

The control unit 200 control the light source 51 of the irradiation optical system 50 to start emitting the laser light for treatment.

### (S21)

The control unit 200 controls the scan optical system 30 according to a predetermined irradiation pattern to irradiate each irradiated position in the irradiation pattern, in which the reference position is arranged at the irradiated target position TP, with the laser light for treatment.

### (S22)

The control unit 200 performs OCT imaging by scanning the irradiated target position TP designated in step S18 (or position(s) of the spot(s) included in the irradiation pattern) or near the position with the measurement light LS from the interference optical system 70.

### (S23)

The control unit 200 causes the image forming unit 240 to form a tomographic image at the irradiated target position TP or near the position TP based on the detection result acquired by the interference optical system 70. The detection result is obtained in step S22. Subsequently, the control unit 200 causes the display of the display unit 230 to display the formed tomographic image.

### (S24)

The control unit 200 stores the image data of the tomographic image, which is displayed on the display of the display unit 230 in step S23, in the storage unit. This terminates the operation of the laser treatment apparatus 1 (END).

### Second Configuration Example

In a second configuration example, in the apparatus optical system 100, the measurement optical system 60 includes an interference optical system 60B. The interference optical system 60B is used for calculating a correction amount for the wavefront correction optical system 40 or for confirming a laser treatment site.

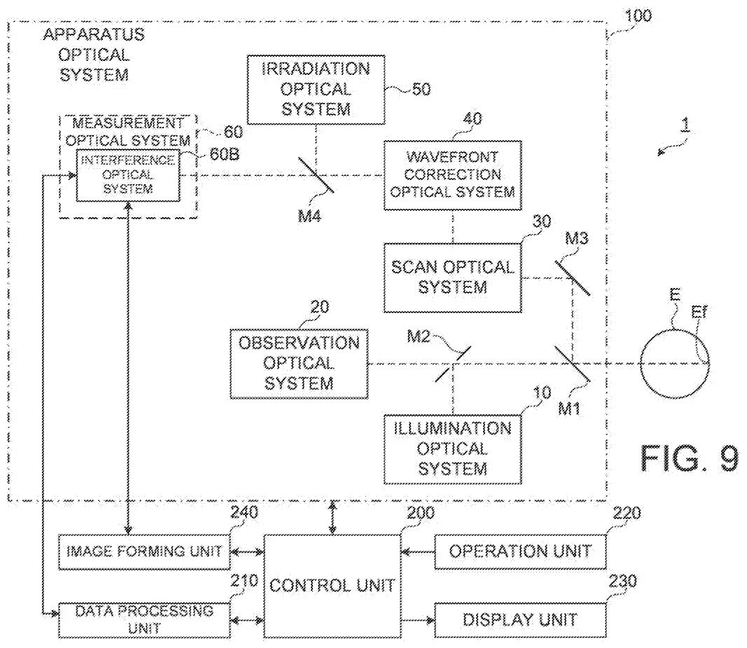

FIG. 9 shows a functional block diagram of the laser treatment apparatus according to the second configuration example of the embodiments. In FIG. 9, parts similar to those in FIG. 1 are denoted by the same reference symbols, and description thereof is omitted as appropriate.

In the apparatus optical system 100 according to the second configuration example, the measurement optical system 60 includes the interference optical system 60B as compared with the apparatus optical system shown in FIG. 1. The configuration of the interference optical system 60B is the same as the configuration of the interference optical system 70 shown in FIG. 4.

Similar to the first configuration example, the laser treatment apparatus 1 according to the second configuration example is provided with the image forming unit 240. The image forming unit 240 forms a tomographic image (OCT image) of the patient's eye E based on the detection result of the interference light acquired by the interference optical system 60B. Specifically, the image forming unit 240 forms image data of the tomographic image of the fundus Ef of the patient's eye E based on the detection result of the interference light obtained by the interference optical system 60B and a pixel position signal input from the control unit 200.

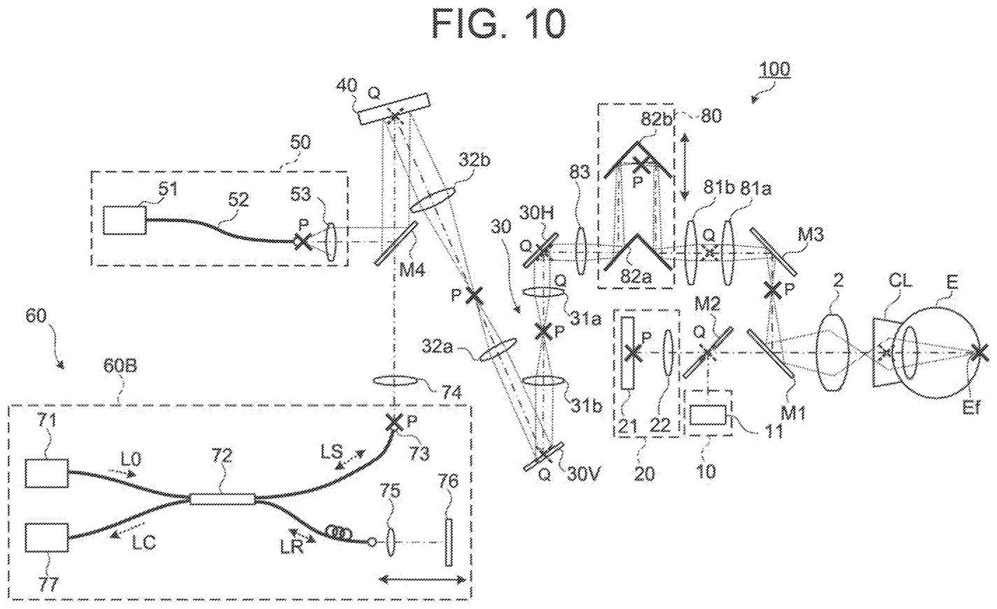

FIG. 10 shows a schematic diagram of the configuration of the optical system of the laser treatment apparatus according to the second configuration example of the embodiments. In FIG. 10, like reference numerals designate like parts as in FIG. 5, and the same description may not be repeated.

The configuration of the optical system shown in FIG. 10 is different from the configuration of the optical system shown in FIG. 5 in that the wavefront measurement optical system 60A and the beam splitter M5 are omitted and that an optical system having the same configuration as that of the interference optical system 70 is provided as the interference optical system 60B.

In the second configuration example, the control unit 200 causes the image forming unit 240 to form the tomographic image based on the measurement result obtained by the interference optical system 60B and causes the display of the display unit 230 to display the formed tomographic image. The control unit 200 causes the data processing unit 210 to calculate the correction amount for the wavefront correction optical system 40 corresponding to the irradiated target position. The irradiated target position is previously determined in the tomographic image displayed on the display or is designated in the tomographic image using the operation unit 220. The control unit 200 can control the wavefront correction optical system 40 based on the calculated correction amount to change the wave front of the laser light for treatment from the irradiation optical system 50.

Alternatively, the control unit 200 may cause the data processing unit 210 to calculate the correction amount for the wavefront correction optical system 40 corresponding to the intensity of the interference light based on the measurement result obtained by the interference optical system 60B. The control unit 200 can control the wavefront correction optical system 40 based on the calculated correction amount to change the wave front of the laser light for treatment from the irradiation optical system 50.

It should be noted that the control unit 200 may display the tomographic image of the laser treatment site before treatment and the tomographic image of the laser treatment site after treatment on the display of the display unit 230. In this case, the laser treatment site can be displayed in each tomographic image in an identifiable manner, or both of the tomographic images can be displayed side by side. Alternatively, the fundus image of the laser treatment site before treatment and the fundus image of the laser treatment site after treatment can be displayed on the display of display unit 230. In this case, the laser treatment site can be displayed in each fundus image in an identifiable manner, or both of the fundus images can be displayed side by side.

### Effects

The effects of the laser treatment apparatus according to the embodiments and the method for controlling the laser treatment apparatus are explained.

A laser treatment apparatus (1) according to some embodiments includes an irradiation system (irradiation optical system 50), a wavefront changing unit (wavefront correction optical system 40), and a controller (control unit 200). The irradiation system is configured to output laser light for treatment from a light source (light source 51). The wavefront changing unit is configured to change a wave front of the laser light for treatment output by the irradiation system to guide the laser light for treatment whose wave front is changed to a patient's eye (E). The controller is configured to control the wavefront changing unit.

According to such a configuration, the wave front of the laser light for treatment is changed by the wavefront changing unit, and the laser light for treatment whose wave front is changed is guided to the patient's eye. Thereby, the focal position of the laser light for treatment can be arranged at an arbitrary position in the patient's eye. Therefore, a laser treatment apparatus capable of reliably irradiating a desired site with the laser light for treatment can be provided. For example, only the desired pyramidal cells of the retina in the patient's eye can be irradiated with laser light for treatment.

The laser treatment apparatus according to some embodiments further includes an optical system (measurement optical system 60) configured to project light onto the patient's eye and to receive returning light from the patient's eye, wherein the controller is configured to control the wavefront changing unit based on light receiving result of the returning light received by the optical system.

According to such a configuration, the state of the patient's eye such as the shape of the fundus or the state of the laser light for treatment such as the beam diameter can be grasped based on the returning light from the patient's eye. Thereby, the wavefront changing unit can be controlled in accordance with the state of the patient's eye or the state of the laser light for treatment. Therefore, the focal position of the laser light for treatment can be arranged in an arbitrary position in accordance with the state of the patient's eye or the state of the laser light for treatment.

In the laser treatment apparatus according to some embodiments, the optical system includes: a lens array (62) configured to generate a plurality of focused light from the returning light; and an area sensor (CCD 61) configured to receive the plurality of focused light generated by the lens array. The laser treatment apparatus further includes a wavefront aberration calculator (data processing unit 210) configured to calculate wavefront aberration of the returning light from the patient's eye based on light receiving result of the plurality of focused light received by the area sensor. The controller is configured to control the wavefront changing unit based on the wavefront aberration calculated by the wavefront aberration calculator.

According to such a configuration, the state of the patient's eye such as the shape of the fundus can be grasped based on the calculated wavefront aberration. Thereby, the wavefront changing unit can be controlled in accordance with the state of the patient's eye. Therefore, the focal position of the laser light for treatment can be arranged in an arbitrary position in accordance with the state of the patient's eye.

In the laser treatment apparatus according to some embodiments, the optical system includes an interference optical system (70, 60B) configured to split light (L0) from a light source (light source unit 71) into reference light (LR) and measurement light (LS), to irradiate the patient's eye with the measurement light, and to detect interference light (LC) between returning light of the measurement light from the patient's eye and the reference light. The laser treatment apparatus includes an interference intensity calculator (data processing unit 210) configured to calculate intensity of the interference light based on detection result of the interference light detected by the interference optical system. The controller is configured to control the wavefront changing unit based on the intensity of the interference light calculated by the interference intensity calculator.

According to such a configuration, the state of the patient's eye such as the shape of the fundus or the state of the laser light for treatment such as the beam diameter can be grasped based on the intensity of the interference light which is generated based on the returning light from the patient's eye. Thereby, the wavefront changing unit can be controlled in accordance with the state of the patient's eye or the state of the laser light for treatment. Therefore, the focal position of the laser light for treatment can be arranged in an arbitrary position in accordance with the state of the patient's eye or the state of the laser light for treatment.

The laser treatment apparatus according to some embodiments further includes an image forming unit (image forming unit 240) configured to form a tomographic image of the patient's eye based on the detection result of the interference light obtained by the interference optical system.

According to such a configuration, whether or not the laser light for treatment has been reliably irradiated to the treatment site can be easily determined, by confirming the tomographic images of the laser treatment site before and after the treatment. Thereby, the effect of laser treatment, which has been difficult to confirm in the past, can be easily grasped.

The laser treatment apparatus according to some embodiments further includes an interference optical system (70, 60B) configured to split light (L0) from a light source (light source unit 71) into reference light (LR) and measurement light (LS), to irradiate the patient's eye with the measurement light, and to detect interference light (LC) between returning light of the measurement light from the patient's eye and the reference light; and an image forming unit (image forming unit 240) configured to form a tomographic image of the patient's eye based on detection result of the interference light obtained by the interference optical system.

According to such a configuration, it is possible to easily determine whether or not the laser light for treatment has been reliably irradiated to the treatment site by confirming the tomographic images of the laser treatment site before and after the treatment. Thereby, the effect of laser treatment, which has been difficult to confirm in the past, can be easily grasped.

The laser treatment apparatus according to some embodiments further includes a designation unit (operation unit 220) configured to designate a irradiated target position (TP) of the laser light for treatment with respect to a front image of the patient's eye and the tomographic image. The controller is configured to control an irradiated position of the laser light for treatment based on the irradiated target position designated by the designation unit.

According to such a configuration, the irradiated target position of the laser light for treatment can be designated as the three-dimensional position. Thereby, a laser treatment apparatus capable of reliably irradiating a desired site designated by a user with the laser light for treatment can be provided.

The laser treatment apparatus according to some embodiments further includes an optical scanner (scan optical system 30) configured to deflect the laser light for treatment. The controller is configured to change an irradiated position of the laser light for treatment in a second direction (X direction, Y direction) intersecting a first direction (Z direction) in which the laser light for treatment travels by controlling the optical scanner.

According to such a configuration, the irradiated position of the laser light for treatment can be two-dimensionally changed in the direction intersecting the traveling direction of the laser light for treatment, by controlling the optical scanner. Thereby, the laser light for treatment can be reliably irradiated a desired site with a simple configuration.

In the laser treatment apparatus according to some embodiments, the wavefront changing unit is disposed on the light source (51) side with respect to the optical scanner.

According to such a configuration, the irradiated position of the laser light for treatment, whose wave front is changed by the wavefront changing unit, can be changed. Thereby, the size of the wavefront changing unit can be reduced, compared with the case where the wave front of the treatment laser beam after deflection using the optical scanner is changed.

The laser treatment apparatus according to some embodiments further includes a diopter correction unit (diopter correction optical system 80) disposed on the patient's eye side with respect to the optical scanner. The controller is configured to change a fundus conjugate position (P) by controlling the diopter correction unit depending on a refractive power of the patient's eye.

According to such a configuration, the focal point of the apparatus optical system can be arranged at a desired position in the patient's eye in accordance with the diopter of the patient's eye. Thereby, a laser treatment apparatus with high measurement accuracy can be provided regardless of the diopter of the patient's eye.

In the laser treatment apparatus according to some embodiments, the controller is configured to change at least one of a focal position of the laser light for treatment and an irradiated position of the laser light for treatment in a second direction (X direction, Y direction) intersecting a first direction (X direction) in which the laser light for treatment travels, by controlling the wavefront changing unit.

According to such a configuration, the focal position can be three-dimensionally changed in the traveling direction of the laser light for treatment or in the direction intersecting the traveling direction, by simply controlling the wavefront changing unit. Thereby, a laser treatment apparatus capable of reliably irradiating a desired site with the laser light for treatment with a simple configuration can be provided.

In the laser treatment apparatus according to some embodiments, the wavefront changing unit includes a deformable mirror.

According to such a configuration, the wave front of the laser light for treatment can be changed using the deformable mirror. Thereby, a laser treatment apparatus capable of reliably irradiating a desired site with the laser light for treatment with a simple configuration can be provided.

A method of controlling a laser treatment apparatus according to some embodiments includes an irradiation step and a wavefront changing step. The irradiation step includes a step of outputting laser light for treatment from a light source (51). The wavefront changing step includes a step of changing a wave front of the laser light for treatment output in the irradiation step to guide the laser light for treatment whose wave front is changed to a patient's eye (E), based on light receiving result of returning light from the patient's eye obtained by irradiating the patient's eye with light.

According to such a method, the wave front of the laser light for treatment output from the light source is changed, and the laser light for treatment whose wave front is changed is guided to the patient's eye. Thereby, the focal position of the laser light for treatment can be arranged at an arbitrary position. Therefore, a laser treatment apparatus can be controlled so as to reliably irradiating a desired site with the laser light for treatment.

The method of controlling the laser treatment apparatus according to some embodiments further includes a wavefront aberration measurement step of measuring a wavefront aberration of the returning light from the patient's eye. The wavefront changing step includes a step of changing a wave front of the laser light for treatment based on the wavefront aberration measured in the wavefront aberration measurement step.

According to such a method, the state of the patient's eye such as the shape of the fundus can be grasped based on the calculated wavefront aberration. Thereby, the wavefront changing unit can be controlled in accordance with the state of the patient's eye. Therefore, the focal position of the laser light for treatment can be arranged in an arbitrary position in accordance with the state of the patient's eye.

The method of controlling the laser treatment apparatus according to some embodiments further includes an interference intensity specifying step of specifying intensity of interference light between returning light of measurement light from the patient's eye and reference light obtained using optical coherence tomography. The wavefront changing step includes a step of changing a wave front of the laser light for treatment based on the intensity of the interference light specified in the interference intensity specifying step.

According to such a method, the state of the patient's eye such as the shape of the fundus or the state of the laser light for treatment such as the beam diameter can be grasped in accordance with the intensity of the interference light generated based on the returning light from the patient's eye. Thereby, the wavefront changing unit can be controlled in accordance with the state of the patient's eye or the state of the laser light for treatment. Therefore, the focal position of the laser light for treatment can be arranged in an arbitrary position in accordance with the state of the patient's eye or the state of the laser light for treatment.

The above-described embodiments are merely examples for carrying out the present invention. Those who intend to implement the present invention can apply any modification, omission, addition, or the like within the scope of the gist of the present invention.

The invention has been described in detail with particular reference to preferred embodiments thereof and examples, but it will be understood that variations and modifications can be effected within the spirit and scope of the invention covered by the claims which may include the phrase “at least one of A, B and C” as an alternative expression that means one or more of A, B and C may be used, contrary to the holding in Superguide v. DIRECTV, 69 USPQ2d 1865 (Fed. Cir. 2004).

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A laser treatment apparatus, comprising:
an irradiation system configured to output laser light for treatment from a light source;
a wavefront changing unit configured to change a wave front of the laser light for treatment output by the irradiation system to guide the laser light for treatment whose wave front is changed to a patient's eye; and
a controller configured to control the wavefront changing unit.

2. The laser treatment apparatus of claim 1, further comprising
an optical system configured to project light onto the patient's eye and to receive returning light from the patient's eye, wherein
the controller is configured to control the wavefront changing unit based on light receiving result of the returning light received by the optical system.

3. The laser treatment apparatus of claim 2, wherein
the optical system comprises:
a lens array configured to generate a plurality of focused light from the returning light; and
an area sensor configured to receive the plurality of focused light generated by the lens array, wherein
the laser treatment apparatus further comprises a wavefront aberration calculator configured to calculate wavefront aberration of the returning light from the patient's eye based on light receiving result of the plurality of focused light received by the area sensor, and
the controller is configured to control the wavefront changing unit based on the wavefront aberration calculated by the wavefront aberration calculator.

4. The laser treatment apparatus of claim 2, wherein
the optical system includes an interference optical system configured to split light from a light source into reference light and measurement light, to irradiate the patient's eye with the measurement light, and to detect interference light between returning light of the measurement light from the patient's eye and the reference light,
the laser treatment apparatus comprises an interference intensity calculator configured to calculate intensity of the interference light based on detection result of the interference light detected by the interference optical system, and
the controller is configured to control the wavefront changing unit based on the intensity of the interference light calculated by the interference intensity calculator.

5. The laser treatment apparatus of claim 4, further comprising
an image forming unit configured to form a tomographic image of the patient's eye based on the detection result of the interference light obtained by the interference optical system.

6. The laser treatment apparatus of claim 1, further comprising
an interference optical system configured to split light from a light source into reference light and measurement light, to irradiate the patient's eye with the measurement light, and to detect interference light between returning light of the measurement light from the patient's eye and the reference light; and
an image forming unit configured to form a tomographic image of the patient's eye based on detection result of the interference light obtained by the interference optical system.

7. The laser treatment apparatus of claim 5, further comprising
a designation unit configured to designate a irradiated target position of the laser light for treatment with respect to a front image of the patient's eye and the tomographic image, wherein
the controller is configured to control an irradiated position of the laser light for treatment based on the irradiated target position designated by the designation unit.

8. The laser treatment apparatus of claim 1, further comprising
an optical scanner configured to deflect the laser light for treatment, wherein
the controller is configured to change an irradiated position of the laser light for treatment in a second direction intersecting a first direction in which the laser light for treatment travels by controlling the optical scanner.

9. The laser treatment apparatus of claim 8, wherein
the wavefront changing unit is disposed on the light source side with respect to the optical scanner.

10. The laser treatment apparatus of claim 8, further comprising
a diopter correction unit disposed on the patient's eye side with respect to the optical scanner, wherein
the controller is configured to change a fundus conjugate position by controlling the diopter correction unit depending on a refractive power of the patient's eye.

11. The laser treatment apparatus of claim 1, wherein
the controller is configured to change at least one of a focal position of the laser light for treatment and an irradiated position of the laser light for treatment in a second direction intersecting a first direction in which the laser light for treatment travels, by controlling the wavefront changing unit.

12. The laser treatment apparatus of claim 1, wherein
the wavefront changing unit includes a deformable mirror.

13. A method of controlling a laser treatment apparatus, comprising:
an irradiation step of outputting laser light for treatment from a light source;
a wavefront changing step of changing a wave front of the laser light for treatment output in the irradiation step to guide the laser light for treatment whose wave front is changed to a patient's eye, based on light receiving result of returning light from the patient's eye obtained by irradiating the patient's eye with light.

14. The method of controlling the laser treatment apparatus of claim 13, further comprising
a wavefront aberration measurement step of measuring a wavefront aberration of the returning light from the patient's eye, wherein
the wavefront changing step includes a step of changing a wave front of the laser light for treatment based on the wavefront aberration measured in the wavefront aberration measurement step.

15. The method of controlling the laser treatment apparatus of claim 13, further comprising
an interference intensity specifying step of specifying intensity of interference light between returning light of measurement light from the patient's eye and reference light obtained using optical coherence tomography, wherein
the wavefront changing step includes a step of changing a wave front of the laser light for treatment based on the intensity of the interference light specified in the interference intensity specifying step.
